(19) United States
(12) Patent Application Publication
(10) Pub. No.: US 2026/0200857 A1
(43) Pub. Date: Jul. 16, 2026

(54) PROCESS FOR THE PRODUCTION OF ALKYL-PYRIDINE N-OXIDES

(71) Applicant: SALTIGO GmbH, Leverkusen (DE)

(72) Inventors: Karsten von dem Bruch, Leverkusen (DE); Andre Grossmann, Cologne (DE); Michael Lee, Burscheid (DE); Roberto Pillon, Kuerten (DE)

(73) Assignee: SALTIGO GmbH, Leverkusen (DE)

(21) Appl. No.: 19/136,920

(22) PCT Filed: Dec. 20, 2023
(86) PCT No.: PCT/EP2023/086879

§ 371 Date: Jun. 9, 2025

(30) Foreign Application Priority Data
Dec. 22, 2022 (EP) 22216063.2

(51) Int. Cl. C07D 213/89

(52) U.S. Cl. CPC C07D 213/89

(57) ABSTRACT

The present invention relates to a process for preparing alkylpyridine N-oxides of formula (I) from the corresponding unsubstituted 3-alkylpyridines or the 5-alkylpyridines correspondingly substituted in the 2 position of formula (II) in the presence of at least oxidizing agent, water, organic solvent and catalyst.

The present invention relates to a process for preparing alkylpyridine N-oxides of formula (I) from the corresponding unsubstituted 3-alkylpyridines or the 5-alkylpyridines correspondingly substituted in the 2 position of formula (II) in the presence of at least oxidizing agent, water, organic solvent and catalyst.

Alkylpyridine N-oxides of formula (I) are reactants for the synthesis of pharmaceutical and agrochemical active ingredients. Such structural elements are found, for example, in acetyl-CoA carboxylase inhibitors in WO2014/114578 A2, which may be used for the treatment of, for example, diabetes or obesity. Furthermore, WO 2014/124230 A2 discloses 5-alkyl-2-halogenylpyridine N-oxides of formula (I) as reactants in the preparation of active ingredients from the group of ERK kinase inhibitors as active ingredients which may be used for the treatment of cancer.

### PRIOR ART

Processes for preparing such compounds are already known from the literature, but these processes have disadvantages when they are carried out on an industrial scale. For instance, oxidations of substituted pyridines with organic peroxides, such as m-chloroperbenzoic acid (MCPBA), are known. The disadvantage here is the amount of m-chlorobenzoic acid produced as waste material, which may have to be laboriously separated from the product. In addition, organic peroxides require high safety measures, particularly when they are stored on an industrial scale.

For oxidation, hydrogen peroxide is a popular oxidizing agent in organic synthesis since the reaction products produced therefrom are only gaseous oxygen and water, which do not have to be laboriously disposed of as waste materials. For example, the use of hydrogen peroxide-urea salts as oxidizing agent is known. However, this procedure has the disadvantage that stoichiometric amounts of waste are produced.

In contrast, the use of an aqueous hydrogen peroxide solution would be advantageous, since this does not produce any waste. Multiple documents have been found that describe the preparation of 5-alkyl-2-halopyridine N-oxides using hydrogen peroxide either in an uncatalyzed manner or in the presence of sodium tungstate (Na₂WO₄)—as described for example in CN103193704A—or trifluoroacetic anhydride. In-house studies have shown that some 5-alkyl-2-halogenylpyridines or 5-alkyl-2-cyanopyridines are however not completely converted in this procedure even in the case of a long reaction time. Independently of this, the reaction in the presence of sodium tungstate and trifluoroacetic anhydride on an industrial scale produces wastes and/or spent catalysts, which have to be laboriously disposed of or reprocessed.

For example, with acetic acid as solvent, WO2005/085248 A1 describes the reaction of 2-chloro-5-methylpyridine with aqueous hydrogen peroxide solution, where the 2-chloro-5-methylpyridine N-oxide was obtained on a gram scale after 8 hours of reaction time in yields of 82 percent. When this process is transferred to an industrial scale, there is the disadvantage that the reaction times would be increased to 24 hours or more and large amounts of acetic acid would have to be laboriously separated from the product and disposed of as waste material in a costly manner. In the case of such reactions, long reaction times also always mean an increased safety risk.

Also known are processes for the oxidation of various monosubstituted substituted pyridines using hydrogen peroxide in the presence of catalysts. For instance, M. R. Prasad, Mol. Cat. A: Chemical 2002, 186, 109-120 describes the oxidation of 2-chloropyridine in water and methanol in yields of 91% and 98%, respectively, using hydrogen peroxide as a 30% aqueous solution in the presence of a TS-1 catalyst at a temperature of 60° C. With water as solvent, the reaction time was 24 hours, while with methanol a reaction time of only 2 hours was sufficient. Analogously, for example 2-, 3- and 4-methylpyridine were obtained in water in yields of only 29% to 32% using hydrogen peroxide as a 30% aqueous solution in the presence of a TS-1 catalyst at a temperature of 60° C. within 24 hours. With methanol as solvent, the methyl-substituted pyridine N-oxides are obtainable within 5 to 6 hours at 60° C. in high yields of 93% to 95% in the presence of a Ti-ZSM-5 (30) catalyst. While halogens act as electron-withdrawing substituents on the pyridine ring, alkyl substituents are electron-donating substituents. According to this literature, pyridine N-oxides with electron-donating substituents can be obtained only in relatively low yields using this method with TS-1 as catalyst. All reactions according to this literature were carried out with 2 molar equivalents of hydrogen peroxide based on the pyridine used and only on a milliliter scale.

There was therefore the need for a process for preparing alkylpyridine N-oxides of formula (I) which makes it possible to prepare these pyridine derivatives efficiently and safely on an industrial scale.

Surprisingly, a simple and safe process for preparing alkylpyridine N-oxides, preferably 3-alkylpyridine N-oxides or 5-alkyl-2-halogenylpyridine N-oxides or 5-alkyl-2-cyanopyridine N-oxides, of formula (I) has been found, which comprises the reaction of alkylpyridines, preferably of 3-alkylpyridines, 5-alkyl-2-halogenylpyridines or 5-alkyl-2-cyanopyridines, of formula (II) with oxidizing agents in the presence of catalysts, water and organic solvents to give these products on an industrial scale in good yields and high purities. In this process, the solid catalyst can be easily separated from the reaction mixture and optionally reused in the process. Moreover, the process according to the invention does not produce any critical waste materials that have to be laboriously disposed of.

The invention therefore provides a process for preparing compounds of formula (I),

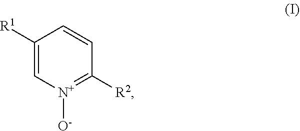

in which R¹ is linear or branched C₁-C₁₀ alkyl, preferably linear or branched C₁-C₆ alkyl, which may be unsubstituted, monosubstituted or polysubstituted, preferably by halogenyl or alkoxy radicals,
or in which R¹ is C₃-C₈ cycloalkyl, which may be unsubstituted, monosubstituted or polysubstituted,
or in which R¹ is aralkyl, which may be unsubstituted, monosubstituted or polysubstituted, preferably by halogenyl or alkoxy radicals,
and in which R² is hydrogen, chlorine, bromine, fluorine or cyano,
comprising at least the reaction of compounds of formula (II),

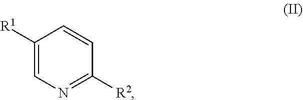

in which the radical R¹ and R² have the definition given for formula (I),
in the presence of at least oxidizing agent, water, organic solvent and catalyst.

Linear C₁-C₁₀ alkyl according to R¹ is for example methyl, ethyl, n-propyl, n-butyl, n-pentyl, n-hexyl, n-heptyl, n-octyl, n-nonyl or n-decyl.

Preferably, unsubstituted linear or branched C₁-C₁₀ alkyl according to radical R¹ is methyl, ethyl, or n-propyl. Preferably, substituted linear alkyl according to radical R¹ is cyclopropylmethyl or 1,1-difluoroethyl.

Linear or branched C₁-C₆ alkyl according to R¹ is for example methyl, ethyl, n-propyl, isopropyl, n-butyl, sec-butyl, isobutyl, n-pentyl, sec-pentyl, 3-pentyl, 2-methylbutyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2,3-dimethyl-2-butyl, 3,3-dimethyl-2-butyl or 3-ethyl-1-butyl.

The linear or branched C₁-C₁₀ alkyl or C₁-C₆ alkyl may be unsubstituted. It may also be monosubstituted or polysubstituted, preferably by halogenyl or alkoxy radicals. Examples of monosubstituted C₁-C₆ alkyl are 2-methoxy-1-ethyl, 2-ethoxy-1-ethyl, 3-methoxy-1-propyl, 3-ethoxy-1-propyl or 1-cyclopropylmethyl, 1-cyclopropylethyl, 1-cyclobutylethyl, 1-cyclopentylethyl, 1-cyclohexylethyl, 2-cyclopropylethyl, 2-cyclobutylethyl, 2-cyclopentylethyl, 2-cyclohexylethyl, 1,1-difluoroethyl or 2,2-difluorocyclopropylmethyl.

C₃-C₈ cycloalkyl according to R′ is for example cyclopropyl, cyclobutyl, cyclopentyl, cyclohexyl, cycloheptyl or cyclooctyl.

C₃-C₈ cycloalkyl may be unsubstituted, monosubstituted or polysubstituted. Examples of monosubstituted C₃-C₈ cycloalkyl are 2-methylcyclobutyl, 3-methylcyclobutyl, 2-methylcyclopentyl, 3-methylcyclopentyl, 2-methylcyclohexyl, 3-methylcyclohexyl, 4-methylcyclohexyl, 2-methylcycloheptyl, 3-methylcycloheptyl, 4-methylcycloheptyl, 2-ethylcyclobutyl, 3-ethylcyclobutyl, 2-ethylcyclopentyl, 3-ethylcyclopentyl, 2-ethylcyclohexyl, 3-ethylcyclohexyl, 4-ethylcyclohexyl, 2-propylcyclobutyl, 3-propylcyclobutyl, 2-propylcyclopentyl, 3-propylcyclopentyl, 2-butylcyclobutyl, 3-butylcyclobutyl, 2-hydroxycyclopropyl, 2-fluorocyclopropyl.

Particularly preferably, R¹ is methyl, ethyl and n-propyl and R² is hydrogen. Likewise particularly preferably, R¹ is methyl, ethyl and n-propyl and R² is chlorine. Likewise particularly preferably, R¹ is methyl, ethyl and n-propyl and R² is bromine. Likewise particularly preferably, R¹ is methyl, ethyl and n-propyl and R² is fluorine. Likewise particularly preferably, R¹ is methyl, ethyl and n-propyl and R² is cyano.

According to the invention, “process for preparing compounds of formula (I) by the reaction of compounds of formula (II) in the presence of at least oxidizing agent, water, organic solvent and catalyst” means that compounds of formula (II) are oxidized with the oxidizing agent by bringing them into contact with catalyst in water and organic solvent to give compounds of formula (I).

In the process according to the invention, the catalyst is for example a microporous solid, preferably a titanium silicalite, particularly preferably titanium silicalite-1, also called TS-1. TS-1 is commercially available from various manufacturers.

Microporous solids are for example molecular sieves such as ZSM5. ZSM5 is an aluminosilicate zeolite belonging to the pentasil family. It is constructed from silicon cations, aluminum cations, oxygen cations and optionally further countercations.

Titanium-containing silicalites have the general chemical formula Si₁₋ₓTiₓO₂ and are prepared for example in a hydrothermal reaction of tetraethyl silicate (TES) as SiO₂ source with tetraethyl titanate (TET) as TiO₂ source in the presence of tetrapropylammonium hydroxide as base. Following the reaction is usually a calcination in order to remove ammonium salts from the solid. This produces porous solids in which tetravalent Si(IV) species are isomorphically replaced by tetravalent Ti(IV) species.

The titanium silicalite most often used on an industrial scale is titanium silicalite-1, also commonly abbreviated to TS-1. This generally has a molar SiO₂/TiO₂ ratio of at least 25, a BET surface area of 360 to 420 g/m² and a pore diameter of approx. 0.5 nanometer. Titanium silicalite-1 or TS-1 is particularly preferred as catalyst for the process according to the invention.

Very particularly preferably, the process according to the invention takes place in the presence of 7 to 15 grams of catalyst per mole of compound of formula (II) used in the process. In particular, the process according to the invention takes place in the presence of 11 to 13 grams of catalyst per mole of compound of formula (II) used in the process. According to the invention, “in the presence of catalyst” means that the reaction mixture containing compound of formula (II), oxidizing agent, water and organic solvent is brought into contact in the liquid phase with catalyst, with the result that the reaction mixture is in the form of a suspension. This suspension is usually mixed mechanically or hydraulically in order to increase the interaction of the liquid phase with the suspended catalyst.

In the process according to the invention, the oxidizing agent is preferably hydrogen peroxide. Hydrogen peroxide is usually used as oxidizing agent in the form of aqueous solutions. In the process according to the invention, the oxidizing agent is particularly preferably a 30 to 70 percent (based on % by weight) aqueous solution of hydrogen peroxide. Particularly preferably, in the process according to the invention, from 0.9 to 2.0 mol, in particular from 1.0 to 1.3 mol, of oxidizing agent is used per mole of compound of formula (II) used.

The process according to the invention is further carried out in the presence of water. In addition to the water present in the aqueous hydrogen peroxide solution, it is possible here for example to also add further water to the reaction mixture. However, preference is given to not adding any additional water to the reaction mixture other than the water present in the oxidizing agent, preferably hydrogen peroxide.

The process according to the invention is further carried out in the presence of at least one organic solvent. The catalyst is present as a solid in the reaction mixture, while the compounds of formula (I) and formula (II) are usually partially or completely dissolved in the solvent. Preferred organic solvents are linear aliphatic alcohols, particularly preferably methanol, ethanol, 1-propanol or 1-butanol, or any desired mixtures thereof. Methanol is particularly preferred as solvent for the process according to the invention. This has the advantage that the temperature of the reaction mixture is limited by the boiling point of the methanol at the respective pressure in the reaction vessel, which can prevent decomposition of the oxidation product.

In an alternative preferred embodiment, the process according to the invention is further carried out in the presence of organic acids, for example acetic acid. The presence of organic acids, for example acetic acid, in addition to the presence of oxidizing agent, water, organic solvent and catalyst has the advantage that, firstly, the reaction proceeds more quickly and, secondly, the acetic acid stabilizes the pyridine N-oxide during the workup, in which thermal stress occurs. Particularly preferably, the process according to the invention is carried out in the presence of 1.0 to 3.0 mol of acetic acid per mole of compound of formula (II) used, in particular in the presence of 1.0 to 1.2 mol of acetic acid per mole of compound of formula (II) used.

In a further preferred embodiment, the process according to the invention is further carried out in the presence of at least one inorganic base. The presence of inorganic base has the advantage that the formation of alkylcarboxylic esters, for example from acetic acid and methanol, is largely suppressed by increasing the pH.

The inorganic base is preferably selected from alkali metal hydroxides, alkaline earth metal hydroxides, alkali metal carbonates or alkaline earth metal carbonates, alkali metal hydrogencarbonates or alkaline earth metal hydrogencarbonates. Particularly preferably, the inorganic base is sodium hydroxide and sodium hydrogencarbonate. In the process according to the invention, the base may be used either in solid or liquid form, as pure substance, or in a form suspended or dissolved in liquid media.

Particularly preferably, the process according to the invention is carried out in the presence of 0.001 to 0.1 mol of inorganic base per mole of compound of formula (II) used, in particular in the presence of 0.005 to 0.05 mol of inorganic base per mole of compound of formula (II) used.

In the process according to the invention, the compounds of formula (II) are reacted for example at temperatures of 60° C. to 90° C., preferably of 70° C. to 85° C.

Preferably, the process according to the invention is carried out in such a way that
a) at least the compound of formula (II) and optionally organic solvent are initially charged, and
b) the mixture from step a) is heated to a temperature of 60° C. to 90° C., preferably of 70° C. to 85° C., and
c) the oxidizing agent is added to the mixture from step b) over the course of 1 to 7 hours, preferably 1 to 5 hours.

Preferably, it is possible here to first mix the starting materials—except the oxidizing agent—i.e. compound of formula (II), catalyst, organic solvent, optionally water, optionally acetic acid, optionally inorganic base, initially individually or together, either as pure substance or dissolved or suspended, at ambient temperature.

Likewise preferably, the process according to the invention is carried out in such a way that
a) at least the compound of formula (II), optionally oxidizing agent and optionally organic solvent are initially charged, and
b) the mixture from step a) is heated to a temperature of 60° C. to 90° C., preferably of 70° C. to 85° C., and
c) the oxidizing agent is added to the mixture from step b) over the course of 1 to 7 hours, preferably 1 to 5 hours.

Likewise preferably, it is possible to first mix the starting materials, i.e. compound of formula (II), catalyst, organic solvent, optionally water, optionally acetic acid, optionally inorganic base, and a portion of the oxidizing agent required for the reaction, initially individually or together, either as pure substance or dissolved or suspended, at ambient temperature.

Subsequently, the reaction mixture is preferably heated to the required reaction temperature. Likewise preferably, the reaction mixture is heated to boiling heat under reflux. The oxidizing agent, preferably hydrogen peroxide, is then added to the mixture of the starting materials that has been brought to reaction temperature.

If a portion of the oxidizing agent has already been added to the reaction in step a), the remainder of the oxidizing agent required for the reaction and not yet added in step a) is added in step c) over the course of 1 to 7 hours, preferably over the course of 1 to 5 hours.

The oxidizing agent is preferably added under temperature control, since the reaction proceeds exothermically. This may involve cooling the reaction mixture. Particularly preferably, the oxidizing agent is added continuously. The oxidizing agent is usually added to the mixture of the starting materials over the course of 1 to 10 hours, preferably of 2 to 5 hours. The metering of oxidizing agent into the reaction mixture, which contains, inter alia, the catalyst, likewise has the advantage that no temporary excesses of hydrogen peroxide in the reaction mixture are required. This makes it possible for the reaction to be handled safely even on a large scale, since an accumulation of the peroxide in the reaction mixture is not to be expected at any time.

Once addition of oxidizing agent has ended, the reaction mixture is preferably kept at the temperatures of 60° C. to 90° C., preferably of 70° C. to 85° C., until there is no further reaction. The chemical reaction is usually monitored by gas chromatography, thin-layer chromatography, infrared spectroscopy or HPLC.

The process according to the invention is usually carried out at ambient pressure or at a pressure of up to 0.6 megapascal under inert gas, for example nitrogen or argon. The process according to the invention produces molecular oxygen, which escapes from the reaction mixture in gaseous form on account of the reaction temperature and can be discharged from the reaction circuit for safety reasons.

If the reaction temperature is above the boiling point of the reaction mixture or of the individual components of the reaction mixture at ambient pressure, the reaction is usually carried out in pressure-tight apparatuses, for example in autoclaves, under elevated autogenous pressure or under applied pressures, for example of nitrogen.

After the end of the reaction of the compound of formula (II), the reaction product, i.e. the compounds of formula (I), is obtained from the reaction mixture for example by
i) filtering the reaction mixture which has cooled to ambient temperature, preferably to from 30° C. to 50° C., with the catalyst being separated from the filtrate, and
ii) optionally washing the filtered-off catalyst with organic solvent, with a wash liquor being obtained, and
iii) mixing filtrate from step a) and wash liquor from step b), and
iv) isolating the reaction product, i.e. compound of formula (I), by separating it from the organic solvent, optionally from water and/or acetic acid, for example by distillation, and
v) drying filtered-off catalyst optionally with a stream of inert gas and optionally reusing it in a subsequent reaction.

The compound of formula (I) may be stored in pure form or in the form of solutions in organic solvents or acetic acid and/or be used as reactant in a subsequent process. It should generally be noted that pyridine N-oxides can be thermally unstable substances. Therefore, appropriate safety investigations and precautionary measures should be taken when handling them on an industrial scale.

Surprisingly, what has hereby been found is a safe, quick and economical process for preparing compounds of formula (I) which overcomes the disadvantages of the processes according to the prior art. Even on an industrial scale, for example with 1000 to 2000 kg of compound of formula (II) as starting material, the yield of compound of formula (I), based on compound of formula (II) used, of the process according to the invention is between 90 and 99 percent of theory at reaction times of below 10 hours. The compound of formula (I) is formed here with a high purity, for example with less than 0.5% by weight of reactant and/or secondary components. Spent catalyst may optionally be reused in a subsequent reaction. Moreover, significantly fewer waste materials are formed in the process according to the invention than in the processes according to the prior art.

### EXAMPLES

### Example 1: Preparation of 2-chloro-5-methylpyridine 1-oxide (Comparative Example)

A mixture of 50 g (0.39 mol) of 2-chloro-5-methylpyridine, 70 g of water and 2.5 g of titanium silicalite TS-1 was heated to 60° C. 53 g (0.78 mol) of hydrogen peroxide (aqueous solution, 50% by weight of H₂O₂) was metered in at 60° C. in 1 hour. Stirring is then continued at 60° C. for 24 hours, after which approx. 50% conversion was achieved.

### Example 2: Preparation of 3-methylpyridine 1-oxide (Inventive)

A mixture of 150 g (1.61 mol) of 3-methylpyridine, 200 g of methanol, 75 g (1.25 mol) of acetic acid and 20 g of titanium silicalite TS-1 was heated to reflux. 130 g (1.91 mol) of hydrogen peroxide (aqueous solution, 50% by weight of H₂O₂) was metered in under reflux in 2 hours. After cooling to ambient temperature, the solid was isolated by filtration and washed with methanol. The combined mother and wash liquors were concentrated under reduced pressure at approx. 40° C. 246 g of a light beige solution (content 66.8% by weight of 3-methylpyridine 1-oxide, yield 93.5% of theory) was obtained as distillation residue.

### Example 3: Preparation of 2-chloro-5-methylpyridine 1-oxide (Inventive)

A mixture of 50 g (0.4 mol) of 2-chloro-5-methylpyridine, 100 g of methanol and 5 g of titanium silicalite TS-1 was heated to reflux. 30 g (0.4 mol) of hydrogen peroxide (aqueous solution, 50% by weight of H₂O₂) was metered in under reflux in 1 hour. Stirring was then continued under reflux for 5 hours. After cooling to ambient temperature, the solid was isolated by filtration and washed with 25 g of methanol. 185 g of a light beige solution (content 28% by weight of 2-chloro-5-methylpyridine 1-oxide, yield 92.1% of theory) was obtained as combined filtrates.

### Example 4: Preparation of 2-chloro-5-methylpyridine 1-oxide (Inventive)

A mixture of 50 g (0.39 mol) of 2-chloro-5-methylpyridine, 100 g of methanol and 5 g of titanium silicalite TS-1 was admixed with 0.1 g of 50% sodium hydroxide solution (0.13 mol) and then heated to reflux. 30 g (0.44 mol) of hydrogen peroxide (aqueous solution, 50% by weight of H₂O₂) was metered in under reflux in 1 hour. Stirring was then continued under reflux for 3 hours. After cooling to ambient temperature, the solid was isolated by filtration and washed with 25 g of methanol. 190 g of a light beige solution (content 27% by weight of 2-chloro-5-methylpyridine 1-oxide, yield 91.2% of theory) was obtained as combined filtrates.

### Example 5: Preparation of 2-chloro-5-methylpyridine 1-oxide (Inventive)

A mixture of 188 g (1.5 mol) of 2-chloro-5-methylpyridine, 244 g of methanol, 94 g of acetic acid and 17 g of titanium silicalite TS-1 was heated to reflux. 107 g (1.6 mol) of hydrogen peroxide (aqueous solution, 50% by weight of H₂O₂) was metered in under reflux in 2 hours. Stirring was then continued under reflux for 2 hours. After cooling to ambient temperature, the solid was isolated by filtration and washed with methanol. The combined mother and wash liquors were concentrated under reduced pressure at approx. 40° C. 242 g of a yellow solution (content 82.7% by weight of 2-chloro-5-methylpyridine 1-oxide, yield 94.6% of theory) was obtained as distillation residue.

### Example 6: Preparation of 2-chloro-5-methylpyridine 1-oxide (Inventive)

A mixture of 1155 g (9.06 mol) of 2-chloro-5-methylpyridine, 1502 g of methanol, 578 g (9.62 mol) of acetic acid, 5.8 g (0.07 mol) of sodium hydrogencarbonate and 105 g of titanium silicalite TS-1 was heated to 76° C. to 78° C. under reflux with introduction of nitrogen. 659 g (9.68 mol) of hydrogen peroxide (aqueous solution, 50% by weight of H₂O₂) was metered in under reflux in 4 hours. Stirring was then continued under reflux for 4 hours. After cooling to 50° C., the solid was isolated by filtration and washed with 115 g of methanol heated to 50° C. The combined mother and wash liquors were concentrated under reduced pressure at approx. 40° C. 1719 g of a red-brown solution (content 72.6% by weight of 2-chloro-5-methylpyridine 1-oxide, yield 96.0% of theory) was obtained as distillation residue.

1. A process for preparing compounds of formula (I), 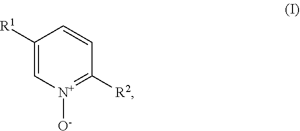
in which R¹ is linear or branched C₁-C₁₀ alkyl, which may be unsubstituted, monosubstituted or polysubstituted,
or in which R¹ is C₃-C₈ cycloalkyl, which may be unsubstituted, monosubstituted or polysubstituted,
or in which R¹ is aralkyl, which may be unsubstituted, monosubstituted or polysubstituted, and in which R² is hydrogen, chlorine, bromine, fluorine or cyano,
comprising at least the reaction of compounds of formula (II), 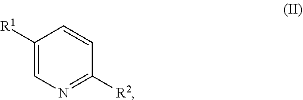
in which R¹ and R² have the definition given for formula (I),
in the presence of at least oxidizing agent, water, organic solvent and catalyst.

2. The process as claimed in claim 1, wherein R¹ is selected from the group consisting of methyl, ethyl and n-propyl and R² is hydrogen, or R¹ is selected from the group consisting of methyl, ethyl and n-propyl and R² is chlorine, or R¹ is selected from the group consisting of methyl, ethyl and n-propyl and R² is bromine, or R¹ is selected from the group consisting of methyl, ethyl and n-propyl and R² is fluorine, or R¹ is selected from the group consisting of methyl, ethyl and n-propyl and R² is cyano.

3. The process as claimed in claim 1, wherein R¹ is cyclopropylmethyl or 1,1-difluoroethyl.

4. The process as claimed in claim 1, wherein the catalyst is a titanium silicalite.

5. The process as claimed in claim 4, wherein the catalyst is titanium silicalite-1 or TS-1.

6. The process as claimed in claim 1, wherein the reaction is effected in the presence of 7 to 15 g of the catalyst per mole of the compound of formula (II).

7. The process as claimed in claim 1, wherein the oxidizing agent is hydrogen peroxide.

8. The process as claimed in claim 1, wherein from 0.9 to 2.0 mol, of the oxidizing agent is used per mole of the compound of formula (II).

9. The process as claimed in claim 1, wherein the reaction is carried out in the presence of organic solvents.

10. The process as claimed in claim 1, wherein the reaction is carried out in the presence of acetic acid.

11. The process as claimed in claim 1, wherein the reaction is carried out in the presence of inorganic base.

12. The process as claimed in claim 11, wherein the inorganic base is selected from alkali metal hydroxides, alkaline earth metal hydroxides, alkali metal carbonates or alkaline earth metal carbonates, alkali metal hydrogencarbonates or alkaline earth metal hydrogencarbonates.

13. The process as claimed in claim 12, wherein the inorganic base is sodium hydroxide or sodium hydrogencarbonate.

14. The process as claimed in claim 1, wherein the reaction is effected at temperatures of 60° C. to 90° C.

15. The process as claimed in claim 1, wherein
a) at least the compound of formula (II) and optionally organic solvent are initially charged, and
b) the mixture from step a) is heated to a temperature of 60° C., and
c) the oxidizing agent is added to the mixture from step b) over the course of 1 to 7 hours.
